(19) United States
(12) Patent Application Publication
(10) Pub. No.: US 2026/0077215 A1
(43) Pub. Date: Mar. 19, 2026

(54) CONTACTLESS, REDUNDANT POSITION DETECTION OF PARALLEL AXES IN MEDICAL DEVICES

(71) Applicant: Varian Medical Systems, Inc., Palo Alto, CA (US)

(72) Inventors: Rolf SCHMIDT, Immenreuth (DE); Leon FICHTL, Eschenbach In Der Oberpfalz (DE); Mario BECHTOLD, Hemhofen (DE)

(73) Assignee: Varian Medical Systems, Inc., Palo Alto, CA (US)

(21) Appl. No.: 18/884,617

(22) Filed: Sep. 13, 2024

(51) Int. Cl. A61N 5/10

(52) U.S. Cl. CPC A61N 5/1049; A61N 5/1045; A61N 5/1067; A61N 2005/105

(57) ABSTRACT

A position control system includes contactless sensors and a controller. The sensors generate sensing signals indicating sensed movement of multiple parts configured to travel along multiple axes, and transmit the sensing signals to the controller, which includes a processor and associated memory. The controller receives, from a first sensor, a first sensing signal indicating sensed movement of parts in a first subset, and receives from a second sensor a second sensing signal indicating sensed movement of parts in a second subset. The two subsets include at least one part in common. A first sensed position of the common part(s) is determined based on the first sensing signal. A second sensed position of the common part(s) is determined based on the second sensing signal. A control signal configured to control movement of the common part(s) is generated based on the first sensed position and the second sensed position.

### TECHNICAL FIELD

This application relates generally to position detection, and more specifically to contactless, redundant position detection of parallel axes in medical devices.

### BACKGROUND

Accurate position detection of movable parts in medical devices may be critical to the success of a medical procedure. In various medical devices, two or more redundant measurement devices are used, with each of the redundant measurement devices being dedicated to detecting and/or measuring a position or movement of a single movable part. Examples of such measurement devices include linear potentiometers and drive unit encoders associated with individual movable parts.

### SUMMARY

The scope of protection sought for various example embodiments is set out by the independent claims. The example embodiments and/or features, if any, described in this specification that do not fall under the scope of the independent claims are to be interpreted as examples useful for understanding various embodiments.

As described in more detail subsequently herein, a robust position sensing, detection, and/or control systems according to some example embodiments, employ contactless sensors that sense movement and/or positions of multiple movable parts concurrently, as opposed to using a single sensor dedicated to detecting the movement of an individual movable part. In some such systems, a first sensor senses movement of a first set of two or more movable parts, and a second sensor senses movement of a different set of movable parts that includes at least one movable part also included in the first set. A third sensor may provide movement sensing for parts included in either or both the first set and/or the second set, while the first and second sensor provide redundant/overlapping movement sensing for movable parts in the third set.

In this way, redundant sensing may be provided using a reduced number of sensors, and without requiring a one-to-one correspondence between sensor and movable part. For example, two sensors may be sufficient to provide redundant sensing for two separate movable parts, in contrast with using a primary and backup sensor dedicated to each movable part, which would require four sensors.

In some example embodiments, a position control system comprises: a plurality of contactless sensors configured to generate sensing signals indicating sensed movement of a plurality of parts configured to travel along a plurality of axes, and transmit the sensing signals to a controller. The controller includes a processor and associated memory configured to receive a first sensing signal from a first contactless sensor, the first sensing signal indicating sensed movement of particular parts included in a first subset of the plurality of parts, receive a second sensing signal from a second contactless sensor, the second sensing signal indicating sensed movement of a second subset of the plurality of parts, the second subset of the plurality of parts including at least one part included in both the first subset and the second subset, determine a first sensed position of the at least one part included in both the first subset and the second subset based on the first sensing signal, determine a second sensed position of the at least one part included in both the first subset and the second subset based on the second sensing signal, and generate a control signal configured to control movement of the at least one part included in both the first subset and the second subset based on the first sensed position and the second sensed position.

In some example embodiments, a leaf collimator comprises: a carriage box; a plurality of collimator leaves moveably mounted in the carriage box; a plurality of drives coupled to individual collimator leaves of the plurality of collimator leaves, the plurality of drives and configured to independently move the individual collimator leaves along a plurality of parallel axes; and a plurality of contactless sensors mounted to the carriage box and configured to generate a first sensing signal indicating sensed movement of a first subset of the plurality of collimator leaves, generate a second sensing signal indicating sensed movement of a second subset of the plurality of collimator leaves, the second subset of the plurality of collimator leaves including at least one collimator leaf included in both the first subset and the second subset, and transmit the first sensing signal and the second sensing signal to a controller, the controller configured to control movement of the at least one collimator leaf included in both the first subset and the second subset based on the first sensing signal and the second sensing signal.

In other example embodiments, a method comprises: sensing using a plurality of contactless sensors changes in positions of a plurality of parts configured to travel along a plurality of axes, the sensing including redundantly sensing positions of each of the plurality of parts by using at least two sensors to sense changes in positions of particular parts included in overlapping subsets of the plurality of parts; generating a first sensing signal indicating sensed movement of a first subset of the plurality of parts; generating a second sensing signal indicating sensed movement of a second subset of the plurality of parts, the second subset including at least one part also included in the first subset; and transmitting the first sensing signal and the second sensing signal to a controller configured to control movement of the at least one part included in both the first subset and the second subset based on the first sensing signal and the second sensing signal.

Any or all of the above example embodiments, and other example embodiments disclosed herein, may be used in various combinations to detect positions of and/or control movement of parts of a medical device along parallel axes.

### BRIEF DESCRIPTION OF THE DRAWINGS

Example embodiments will become more fully understood from the detailed description given herein below and the accompanying drawings, wherein like elements are represented by like reference numerals. The example embodiments are given by way of illustration only, and thus are not limiting of this disclosure.

FIG. 1 is a pictorial representation of a system for providing medical treatment, in accordance with some example embodiments;

FIG. 2 is a block diagram of a position control system, in accordance with some example embodiments;

FIG. 3 is a flow diagram of a method illustrating sensing position changes, in accordance with some example embodiments;

FIG. 4 is a flow diagram of a method illustrating controlling part movement based on sensed input, in accordance with some example embodiments;

FIG. 5 is a diagram illustrating contactless redundant sensing, in accordance with some example embodiments;

FIG. 6 is a diagram illustrating another example of contactless redundant sensing, in accordance with some example embodiments;

FIG. 7 is a pictorial representation of a single magnetic sensor sensing magnetic fields associated with three collimator leaves, in accordance with some example embodiments;

FIG. 8 is a block diagram of magnetic portions of collimator leaves including multiple North and South pole pairs, in accordance with some example embodiments;

FIG. 9 is a block diagram showing multiple magnetic sensors positioned over collimator leaves including multiple North and South magnetic poles, in accordance with some example embodiments;

FIG. 10 is a block diagram of magnetic portions of collimator leaves including single North and South pole pairs, in accordance with some example embodiments;

FIG. 11 is a block diagram showing multiple magnetic sensors positioned over collimator leaves including single North and South pole pairs, in accordance with some example embodiments;

FIG. 12 is a block diagram illustrating a capacitive sensor arrangement, in accordance with some example embodiments;

FIG. 13 is a pictorial representation of a leaf collimator box illustrating placement of collimator leaves in relationship to sensor arrangements, in accordance with some example embodiments;

FIG. 14 is a generalized representation of leaf support system illustrating a location of a position detection system, in accordance with some example embodiments;

FIG. 15 is a perspective view of a leaf collimator support, in accordance with some example embodiments;

FIG. 16 is a picture of a collimator leaf and associated motive system, in accordance with some example embodiments;

FIG. 17 is diagram of a collimator leaf including multiple North and South pole pairs, in accordance with some example embodiments;

FIG. 18 is diagram of a collimator leaf including a single North and South pole pair, in accordance with some example embodiments;

FIG. 19 is a diagram of collimator leaves including scales capable of being optically sensed, in accordance with some example embodiments; and

FIG. 20 is a diagram of collimator leaves including and reflected-energy sensors, in accordance with some example embodiments;

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. The use of similar or identical reference numbers in the various drawings is intended to indicate the presence of a similar or identical element or feature.

### DETAILED DESCRIPTION

Various example embodiments will now be described more fully with reference to the accompanying drawings in which some example embodiments are shown.

Detailed illustrative embodiments are disclosed herein. However, specific structural and functional details disclosed herein are merely representative for purposes of describing example embodiments. It should be understood that there is no intent to limit example embodiments to the particular forms disclosed. On the contrary, example embodiments are to cover all modifications, equivalents, and alternatives falling within the scope of this disclosure. Like numbers may refer to like elements throughout the description of the figures. One or more example embodiments described herein may be combined.

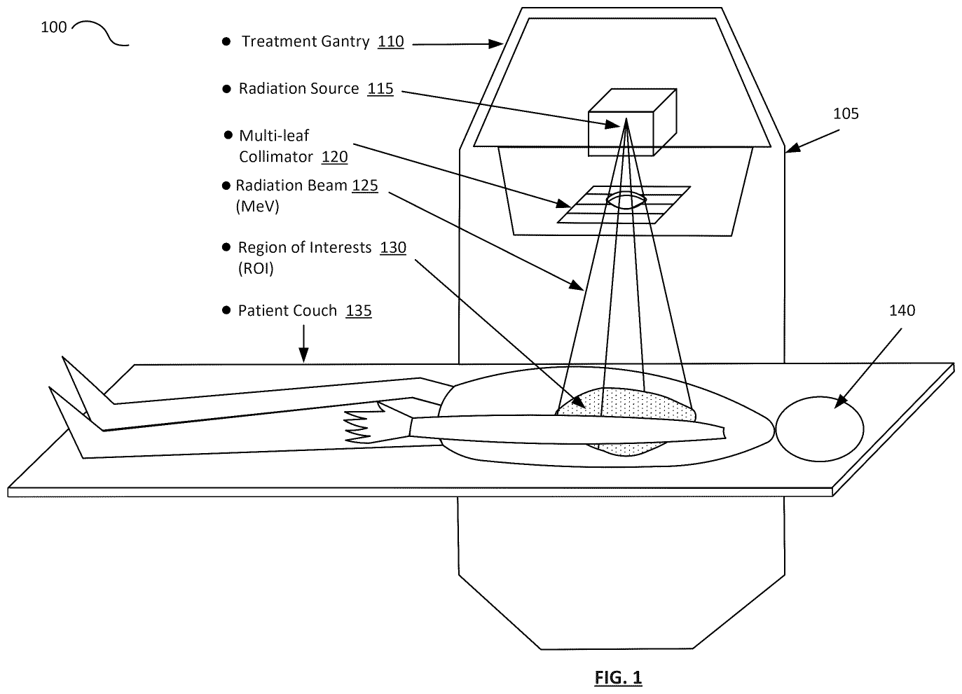

Referring first to FIG. 1, a system 100 for providing medical treatment will be discussed in accordance with various example embodiments. In the illustrated example embodiments, system 100 includes a patient couch 135, on which a patient 140 is positioned so that the region of interest 130 is properly located within the radiation beam 125. The treatment gantry 110 includes the radiation source 115 and the multi-leaf collimator 120. The radiation source 115 directs the radiation beam 125, through the multi-leaf collimator 120, and towards the region of interest 130. Individual leaves of the multi-leaf collimator 120 are arranged block portions of the radiation beam 125 that fall outside the region of interest 130.

In some example embodiments, the patient couch 135 includes multiple movable parts (not illustrated) used to position the patient couch 135 under the treatment gantry 110 and next to, within, or partially within the treatment unit 105. Furthermore, in some example embodiments the treatment gantry 110 may include movable parts that allow the treatment gantry 110 to be rotated about the patient couch 135 or otherwise moved in relationship to the region of interest 130. Movement of the treatment gantry 110 or the patient couch 135 may cause the region of interest 130 to move with respect to the radiation source 115 and the multi-leaf collimator 120. Changes in the relative position of the region of interest 130 may cause the shape and size of the region of interest to vary, which require individual leaves of the multi-leaf collimator 120 to be moved to block different portions of the radiation beam 125.

Redundant contactless position sensing may be used to sense and control movements of movable parts included in the treatment gantry 110, the treatment unit 105, the patient couch 135, and/or the multi-leaf collimator 120. In other example embodiments, positional changes of movable parts in any suitable type of system employing redundant position sensing may be implemented in accordance with the disclosed contactless, redundant position sensing and control techniques disclosed herein.

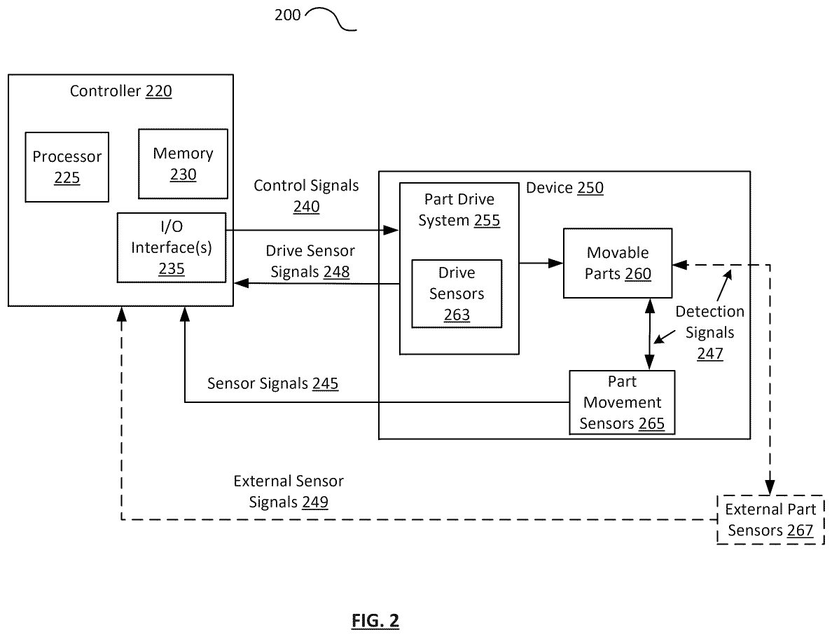

Referring next to FIG. 2, a position control system 200 will be discussed in accordance with various example embodiments. In the illustrated example embodiments, the position control system 200 includes device 250 and controller 220. Device 250 includes a part drive system 255, which may impart motion to the movable parts 260 using mechanical drives, electrical drives, magnetic drives, or the like; movable parts 260, and part movement sensors 265. Controller 220 includes a processor 225, memory 230, and input/output (I/O) interfaces 235.

The part drive system 255 imparts motion to the movable parts 260 along one or more axes. In at least one example embodiment, a set or group of the movable parts 260 move along parallel axes, although at least some example embodiments are not limited to movement along parallel axes. The part drive system 255 also includes drive sensors 263. The drive sensors 263 may include contactless drive sensors that obtain movement and/or position information from which a position of one or more of the moveable parts 260 may be inferred. The contactless drive sensors may include, but are not limited to, magneto-strictive drive-spindle sensors, glass scales, capacitive measurement systems, optical encoders or other visual systems, or the like. In at least one example embodiment, a single magnetic or reflected-light optical sensor may be used to sense rotational or linear movement of multiple drive elements, such as drive spindles, or the like. Furthermore, a single sensor may include a plurality of individual sensing elements. Thus, the term “sensor” may be used herein to refer to either an individual sensing element, or to a collection/group of sensing elements that cooperate perform a sensing function.

In operation, the processor 225 included in the controller 220 generates control signals 240, and transmits those control signals to the part drive system 255 of device 250. Part drive system 255 energizes or actuates a drive mechanism to impart motion to movable parts 260. Changes in position of the movable parts 260 are detected by part movement sensors 265 and/or external part sensors 267 using detection signals 247.

In various example embodiments, the part movement sensors 265 and/or external part sensors 267 are contactless sensors and the detection signals 247 may be considered to be contactless signals. As used herein, the term “contactless,” “contactless sensing,” and similar terms refers to interactions between an object and an electrical signal an electromagnetic signal, such as light, magnetic field(s), soundwaves detectable or undetectable by the human hear, or the like, but without physical contact between two objects.

The part movement sensors 265 and/or the external part sensors 267 transmit sensing signals 245 or external sensor signals 249 to controller 220. Similarly, the drive sensors 263 may transmit drive sensor signals 248 to the controller 220. The controller 220 processes the sensing signals 245, the external sensor signals 249, and/or the drive sensor signals 248 to generate the control signals 240, which are transmitted to the part drive system 255 and used to control movement of the movable parts 260.

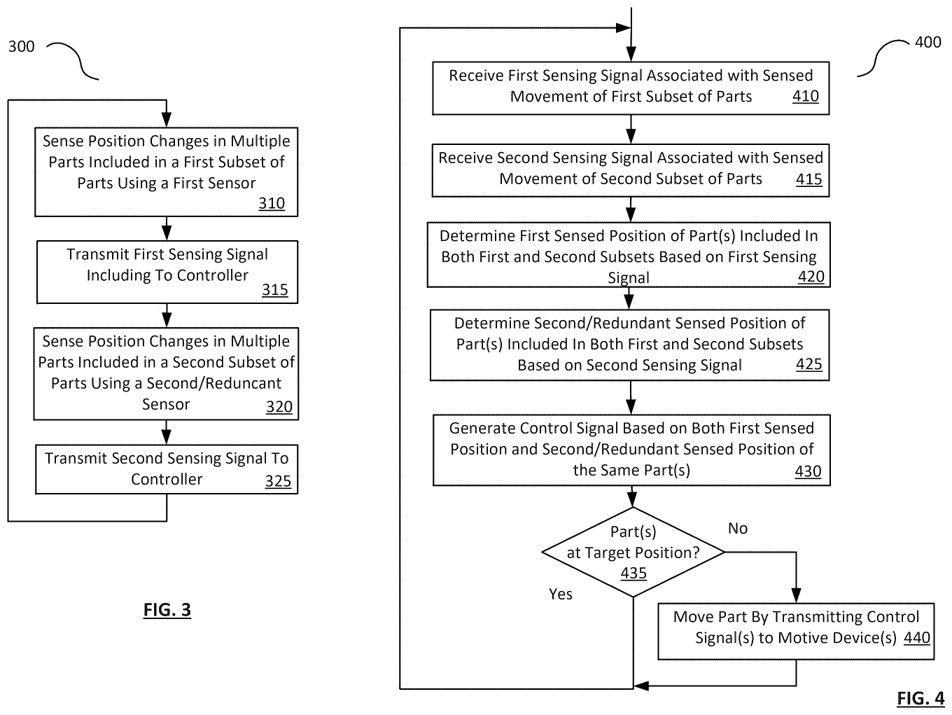

Referring next to FIG. 3, a method 300, which illustrates sensing position changes, will be discussed in accordance with various example embodiments. As illustrated by block 310 position changes of multiple movable parts included in a first subset of movable parts, selected from movable parts 260 (FIG. 2), are sensed using a first sensor, for example one of the part movement sensors 265 (FIG. 2). In at least one example embodiment, the first sensor is a single sensor used to sense movement and/or positions of two or more moveable parts moving along parallel axes. The first sensor may generate a first sensing signal that includes sensing components associated with each of multiple movable parts in the first subset of moveable parts.

For example, a capacitive sensor may be positioned close enough to three plates so that movement of the plates relative to the capacitive sensor causes changes in sensed capacitances associated with each of the plates. A capacitance associated with each plate may contribute to a single sensed capacitance signal. The sensed capacitance signal may include, for example, a current or voltage signal that varies as a function of the capacitance. The sensed capacitance signal may include multiple different components having different frequencies, amplitudes, or the like. Characteristics of the different components may be used to identify contributions of the individual movable parts to the sensed capacitance signal. Sensors other than capacitive sensors may also be used. Examples of other sensors include magnetic field sensors, optical sensors, sonic (e.g. sound-wave) sensors, or the like. Regardless of the exact sensor type, one sensor may sense a change in position or movement of multiple movable objects.

As illustrated by block 315, the first sensor transmits its sensed signal to a controller, such as controller 220 (FIG. 2), for further processing.

As illustrated by block 320, position changes of multiple movable parts included in a second subset of movable parts selected from movable parts 260 (FIG. 2) are sensed using a second sensor, for example one of the part movement sensors 265 (FIG. 2). In at least one example embodiment, the second sensor is a single sensor used to sense movement and/or positions of two or more moveable parts moving along parallel axes. The second sensor may generate a second sensing signal that includes sensing components associated with each of multiple movable parts in the second subset of moveable parts.

As illustrated by block 325, the second sensor transmits its sensed signal to the controller, such as controller 220 (FIG. 2), for further processing.

Note that in various example embodiments, the first subset of movable parts and the second subset of moveable parts include one or more overlapping elements. In an example of the two subsets including multiple overlapping elements or parts, the first subset may include moveable parts A, B, and C, while the second subset of moveable parts includes movable parts B, C, and D. In an example where the two subsets include a single overlapping part, the first subset may include moveable parts A, B, and C, while the second subset of moveable parts includes movable parts C, D, and E. In various example embodiments, the overlapping elements of the two subsets, e.g. one or more movable parts included in both the first and second subsets, allow for redundant movement and/or position sensing.

Referring next to FIG. 4, a method 400 illustrating controlling part movement based on sensed input, will be discussed in accordance with various example embodiments. As illustrated by block 410, a controller, for example, controller 220 (FIG. 2), receives a first sensing signal, for example one of the sensing signals 245 (FIG. 2) associated with sensed movement of a first subset of parts, for example a first subset of movable parts 260 (FIG. 2).

As illustrated by block 415, the controller receives a second sensing signal, for example, another sensing signal included in the sensing signals 245 (FIG. 2). The second sensing signal is associated with sensed movement of a second subset of parts, for example a second subset of movable parts 260 (FIG. 2).

As illustrated by block 420, a first sensed position of part(s) included in both the first and second subsets is determined based on the first sensed sensing signal. For purposes of the present example, assume that the first sensing signal includes movement and/or positional information associated with three movable parts, A, B, and C included in the first subset. Further assume that the second subset may include three movable parts B, C, and D, of which movable parts B and C are common, overlapping, or redundant, to both the first subset and the second subset.

In an example embodiment, the first sensing signal may indicate that moveable part A has changed position by 1 mm along a first axis, that movable part B has changed position by 1.5 mm along a second axis parallel to the first axis, and that movable part C has remained stationary. Using the change in position indicated by the first sensing signal, a first sensed position of each moving part included in the first subset of movable parts may be determined. This includes determining the first sensed positions of the movable parts common to both the first subset and the second subset. In some example embodiments, first sensed positions of the movable parts may be directly sensed, rather than sensing movement of the movable parts and inferring the first sensed position based on the sensed movement. In yet other example embodiments, a combination of sensed positions and sensed movements may be implemented.

As illustrated by block 425 a second sensed position of part(s) included in both the first and second subsets is determined based on the second sensed sensing signal. For example, the second sensing signal may indicate that moveable part B has changed position by 1.5 mm along the second axis, that movable part C has changed position by 0.01 mm along a third axis parallel to the first axis and the second axis, and that movable part D has remained stationary. Using the change in position or a directly sensed position indicated by the first sensing signal, a first sensed position of each moving part included in the first subset of movable parts may be determined.

As illustrated by block 430, the controller generates control signals controlling movement of each of the movable parts B and C based on the first sensing signal and the second sensing signal. The controller also generates control signals to control movement of moveable parts A and D, but those movable parts may be controlled based on different combinations of redundant sensors, and are not explicitly discussed in this example.

Of note in this example, the first sensing signal and the second sensing signal are in agreement regarding the movement of movable part B, but that the first sensing signal indicates that movable part C has remained stationary, while the second sensing signal indicates that moveable part C has changed position by 0.01 mm. With respect to movable part B, where the redundant sensing signals, in this case the first sensing signal and the second sensing signal, are in agreement, the controller may generate a control signal based on the position of redundantly sensed movable part B. The control signal generated for movable part B may indicate forward movement along an axis, backward movement along the axis, or no movement along the axis. The control signal may also indicate a magnitude of the movement, for example a distance to move along the axis. In some example embodiments, the control signal may also control rotational movement about the axis, or movement along an intersecting axis.

As illustrated by block 435, a check is made to determine whether the movable parts have reached their target positions. For example, in at least one example embodiment, a controller may be programmed in advance with a treatment plan indicating target positions for each of the movable parts during particular times during treatment. There may be different target positions during different portions of the treatment plan, so a movable part may reach a first target position at a first point in time, for example during a first iteration of method 400, and then be moved to a second target position at a second point in time, for example during a second iteration of method 400. In some example embodiments, manual input, potentially including signals generated from real time user control of an input device, may also be used as a basis for setting a target position.

In this example, a treatment plan includes one or more target positions of movable parts B and C. Recall that both the first and second sensed positions of movable part B are in agreement, so at block 435 a comparison of the sensed position of movable part B may be compared to a target position of movable part B. If the sensed position of movable part B indicates that movable part B is at the target position, the controller leaves movable part B at its current position.

However, as illustrated by block 440, if the sensed position of movable part B indicates that movable part B is not at the target position, the controller moves movable part B by transmitting a control signal to a motive device attached to movable part B. The motive device may be included in part drive system 255 (FIG. 2).

Referring again to block 430, recall that the first and second sensed positions of movable part C do not exactly match, so the controller may perform processing to arrive at a sensed position to be used in the determination made at block 435. Recall that the first sensed position of movable part C (determined from the first sensing signal generated by a first sensor) indicates that part C did not move. Further recall that the second sensed position of movable part C (determined from the second sensing signal generated by a second sensor) indicates that part C moved by 0.01 mm. The controller may handle this discrepancy in the redundant sensors in various ways.

In some example embodiments, if a difference between two redundant sensors is less than a detection threshold value, the difference can be ignored. For example, assume sensors are capable of detecting movement on the order of 0.1 mm. Any value less than the detection threshold value of 0.1 can be ignored by rounding down. So for example sensed movement of 0.01 mm, would be rounded down to zero, thereby indicating no movement. In other example embodiments, one of the two redundant sensors may be designated as the primary sensor, and for any difference between sensor readings less than the detection threshold, the sensing signal from the designated primary sensor may be used, and the signal from the-non-primary sensor discarded. In yet other embodiments, various statistical combinations can be made, including weighting one sensor's output more heavily than the output of another sensor with regard to particular movable parts. In yet further embodiments, more than two redundant sensors may be used, and a sensed positions obtained from two sensors in agreement can be used for the comparison at block 435.

In some example embodiments, in response to detecting a discrepancy between readings from redundant sensors that falls outside of a predetermined range, the controller may initiate an error handling process including termination of treatment, disabling one or more sensors, flagging readings obtained from one or more sensors, automatically applying an offset to readings from one or more sensors, or the like.

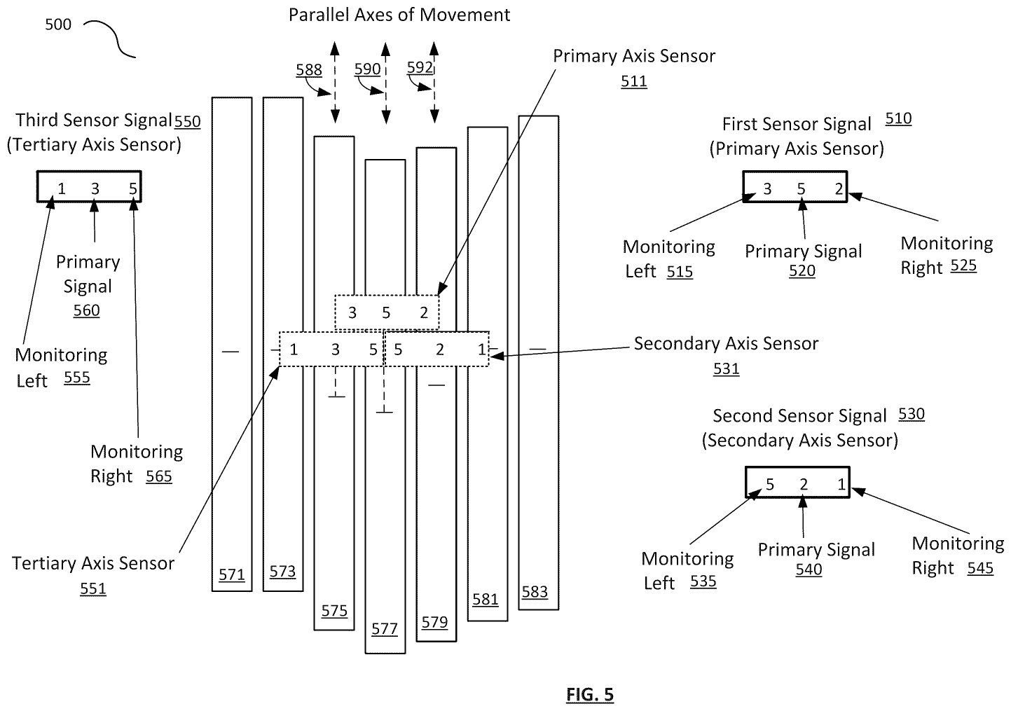

Referring next to FIG. 5, a contactless, redundant sensing arrangement 500, will be discussed in accordance with various example embodiments. The contactless, redundant sensing arrangement 500 includes movable parts 571, 573, 575, 577, 579, 581, and 583 that move along parallel axes. For example, moveable part 577 moves along axis 590, moveable part 579 moves along axis 592, and moveable part 575 moves along axis 588. The contactless, redundant sensing arrangement 500 also includes a primary axis sensor 511 that generates a first sensor signal 510; a secondary axis sensor 531 that generates a second sensor signal 530; and a tertiary axis sensor 551 that generates a third sensor signal 550.

The first sensor signal 510, which is generated by the primary axis sensor 511 includes components indicating movement and/or positions of the movable parts 575, 577, and 579. The second sensor signal 530, which is generated by the secondary axis sensor 531, includes components indicating movement and/or positions of the movable parts 577, 579, and 581. The third sensor signal 550, which is generated by the tertiary axis sensor 551, includes components indicating movement and/or positions of the movable parts 573, 575, and 577. In the illustrated example embodiment, the primary axis sensor 511 is used primarily for sensing changes in position of moveable part 577, but also senses changes in position of movable parts 575 on the left of moveable part 577 and moveable part 579 on the right of moveable part 577. The secondary axis sensor 531 is used primarily for sensing changes in position of moveable part 579, but also senses changes in position of movable parts 577 on the left of moveable part 579 and moveable part 581 on the right of moveable part 579. Similarly, the tertiary axis sensor 551 is used primarily for sensing changes in position of moveable part 575, but also senses changes in position of movable parts 573 on the left of moveable part 575 and moveable part 577 on the right of moveable part 575.

In the illustrated embodiment, the small horizontal lines on movable parts 571, 573, 575, 577, 579, 581, and 583 represent a reference point, for example a change in magnetic polarity, a visual reference mark, or the like. The values within the signal boxes represent a distance from the reference point. The values may be associated with actual distances, may represent signal strengths, or may otherwise vary in a manner that allows a controller to determine a distance moved along one of the parallel axes of movement. For example, first sensor signal 510 includes a left signal component 515 having a value of 3, a right signal component 525 having a value of 2, and a primary signal component having a value of 5. The values of each signal component are associated with a particular moveable part. For example, in embodiments where the movable parts are leaves of a leaf collimator, the primary signal component 520 of first sensor signal 510 may indicate that movable part 577 has moved 5 units from a reference point. Likewise, the left signal component 515 of the first sensor signal 510, may indicate that movable part 575 has moved 3 units from a reference point, and the right signal component 525 of the first sensor signal 510, may indicate that movable part 579 has moved 2 units from a reference point.

Continuing with the illustrated example, the primary signal component 540 of the second sensor signal 530 may indicate that movable part 579 has moved 2 units from a reference point. Likewise, the left signal component 535 of second sensor signal 530, may indicate that movable part 577 has moved 5 units from a reference point, and the right signal component 545 of second sensor signal 530, may indicate that movable part 581 has moved 1 unit from a reference point. In the same way, the primary signal component 560 of the third sensor signal 550 may indicate that movable part 575 has moved 3 units from a reference point. Likewise, the left signal component 555 of third sensor signal 550, may indicate that movable part 573 has moved 1 unit from a reference point, and the right signal component 565 of third sensor signal 550, may indicate that movable part 577 has moved 5 unit from a reference point.

The units represented in the components of the sensor signals may be any suitable unit consistent the scale of movement. If movement is on the scale of mm, then the units may be in mm, and so on. Furthermore, in some embodiments the value of the sensor signal components may be expressed as currents, voltages, resistances, amperes/meter, gauss, lumens, or some measurement consistent with the type of sensors being employed. These measurements may then be converted to sensed positions in a controller to which the sensor signals are transmitted.

Note that in the illustrated example embodiment, all three sensors operate in a redundant fashion, so for example, three separate sensors confirm that movable part 577 has moved 5 units from a reference point. Additional sensors (not illustrated) may be used to provide “three-layer” redundancy for the remaining movable parts. In various example embodiments, additional sensors may be used to provide greater redundancy to even further improve reliability and/or confirmation of sensed measurements. In other implementations, fewer levels of redundancy may be implemented.

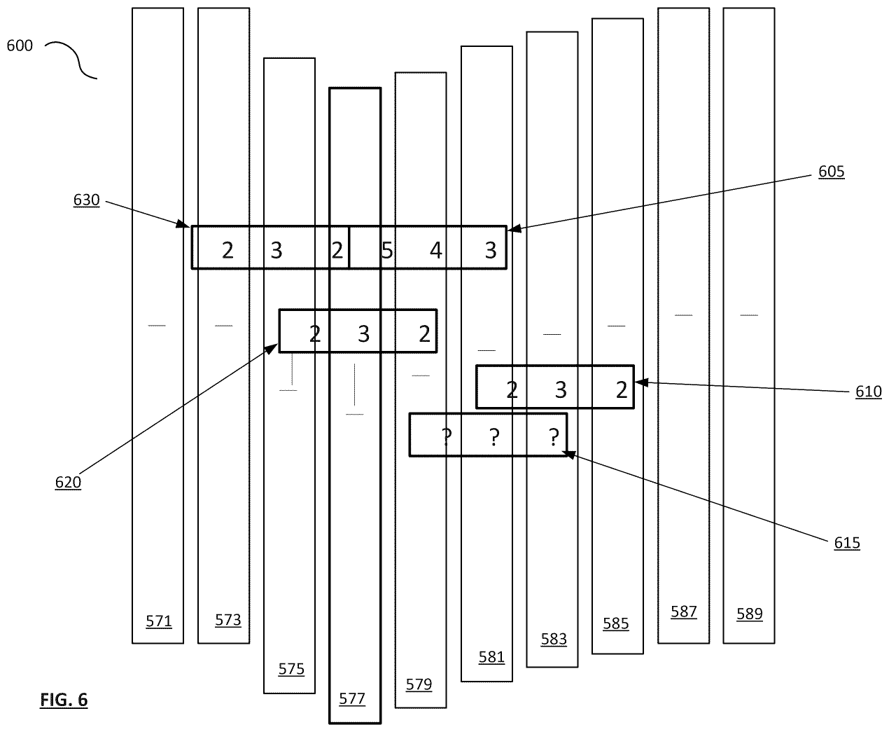

Referring next to FIG. 6, another example of contactless redundant sensing in a sensing system 600 will be discussed in accordance with various example embodiments. Sensing system 600 includes movable parts 571, 573, 575, 579, 581, 583, 585, 587, and 589, along with five sensors 605, 615, 610 620, and 630 that operate in a manner similar to the sensors discussed FIG. 5. Note that in this example embodiment, however, sensor 615 may have failed, leaving some movable parts with a greater number of redundant monitoring sensors than others. For example, movable parts 579 and 581 are left with dual redundancy due to a failure of sensor 615, while movable part 577 is provided with triple redundancy. Other sensors (not illustrated) are assumed to provide triple redundancy to the remaining movable parts. In this example embodiment, failure of a single sensor still allows redundant sensing of the movable parts. It will be appreciated that more or fewer sensors can be employed in accordance with other example embodiments.

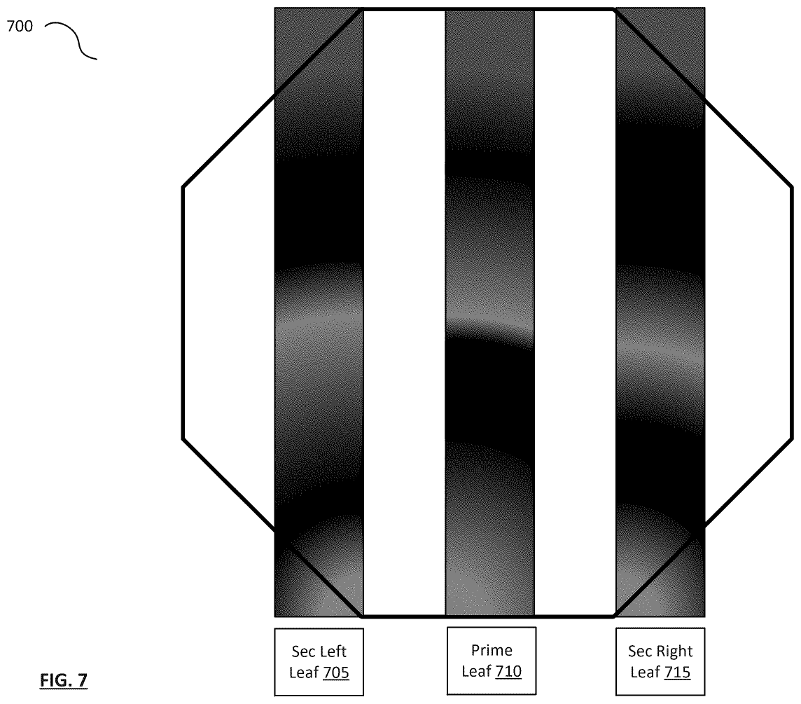

Referring next to FIG. 7, a magnetic sensor 700 used to sense magnetic fields associated with three collimator leaves, will be discussed in accordance with various example embodiments. Magnetic sensor 700 may be positioned to measure magnetic fields associated with a primary leaf 710 and left-secondary leaf 705, and a right-secondary leaf 715. The darker portions of each vertical rectangle represent stronger magnetic fields associated with portions of each leaf over the magnetic sensor 700.

In at least one embodiment, magnetic sensor 700 includes an array of sensing elements sensitive to magnetic fields associated with each of the leaves. In at least one example embodiment, magnetic sensor 700 includes multiple Hall Sensor head. For example, each leaf may include magnetized portions, which may include one or more attached magnets or magnetic strips. In some example embodiments, the magnetic sensor 700 senses only changes in magnetic fields caused by movement of the leaves. In other example embodiments, the strength of a permanent magnetic field may be sensed by the array of sensing elements. Details of constructing basic magnetic sensors of various types are known to those of ordinary skill in the art.

The magnetic sensor 700 may, in some example embodiments, sense a single magnetic field with components of each leaf included in that magnetic field. In some example embodiments the magnetic sensor 700 may differentiate between the magnetic fields of each leaf based on physical proximity of portions of the array of magnetic sensing elements to particular collimator leaves. In either case, the sensor may provide a sensing signal including information indicating a magnetic field strength of primary leaf 710, left-secondary leaf 705, and right-secondary leaf 715 to a controller. The magnetic sensor converts the magnetic field strengths into electrical or other signals suitable for transmission to a controller. Using sensing signals from multiple magnetic sensors covering common collimator leaves, the controller may determine a position, and/or a change in position, of a particular collimator leaf.

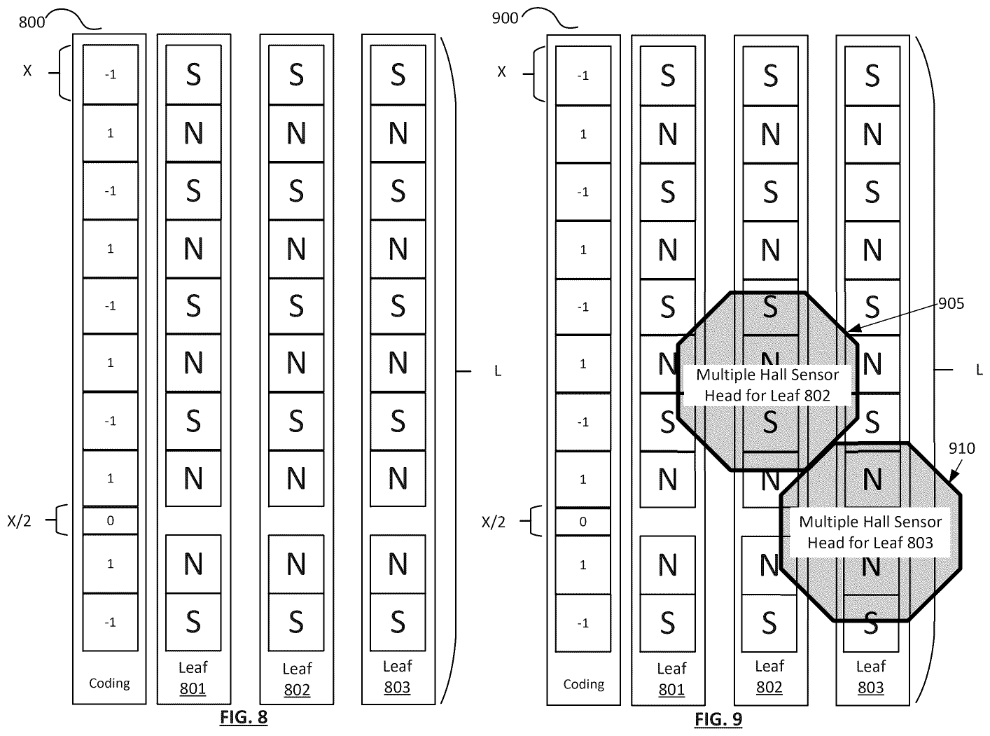

Referring next to FIG. 8 a collimator leaf arrangement 800 will be discussed in accordance with various example embodiments. In the illustrated example embodiments, Collimator leaves 801, 802, and 803 have a length, L, along which multiple groups of North and South magnetic pole pairs are positioned. In collimator leaf arrangement 800, each North pole is coded as “+1” and each South pole is coded as “−1”. A length of each North and South pole has a length represented by “X.” In the illustrated example embodiments, there are four North and South pole pairs in a first group and a single North and South pole pair in a separate group separated by a distance of “X/2.” The space between the groups of North and South pole pairs is coded as “0.” As used herein, the term “coded as” refers to a value output by a magnetic field sensor when it encounters that portion of the collimator leaf. Thus, when a portion of a magnetic sensor array is positioned over a South pole, that portion of the sensor will generate a “−1” value. When a portion of the magnetic sensor array is positioned over a North pole, that portion of the sensor will generate a “+1” value. When a portion of the magnetic sensor array is positioned in the space between the North and South pole pairs, the sensor will return a “0” value.

Referring next to FIG. 9 a magnetic sensing arrangement 900 will be discussed in accordance with various example embodiments. In the illustrated example embodiments, magnetic sensing arrangement 900 includes multiple magnetic sensors, such as Hall sensor heads 905 and 910 positioned over collimator leaves 801, 802, and 803. Hall sensor head 905 may be used as the primary sensor for collimator leaf 802, and to provide backup or redundant sensing for collimator leaves 801 and 803. Hall sensor head 910 may be used as the primary sensor for collimator leaf 803, and to provide backup or redundant sensing for collimator leaf 802. One or more additional Hall sensor heads (not illustrated) may be used to provide primary sensing for collimator leaf 801, and additional redundant sensing for any or all of the collimator leaves 801, 802, and 803.

Note that in magnetic sensing arrangement 900, each of the collimator leaves includes multiple North-South (and South-North) magnetic pole transitions. In at least one example embodiment, Hall sensor heads 905 and 910 detect those magnetic pole transitions and generate a coded sensing component associated with a North pole portion of a collimator leaf at “+1” and generate a coded sensing component associated with a South pole portion of a collimator leaf as “−1.” The part of a collimator leaf associated with neither a North nor South magnetic pole, for example in the area marked “X/2,” may be coded as “0.” The coded sensed values associated with the collimator leaves monitored by each Hall sensor head may be included as components in a composite signal transmitted to a controller. In some example embodiments, the components associated with each monitored collimator leaf may be sent as separate signals to the controller.

In an example of operation, a sensing signal generated by Hall sensor head 905 may include three sensing signal components: a primary component associated with collimator leaf 802, a left component associated with collimator leaf 801, and a right component associated with collimator leaf 803. In the illustrated example, each of the three components may have a value of +1, indicating that the center of Hall sensor head 905 is located over a North magnetic pole.

A sensing signal generated by Hall sensor head 910 may include two sensing signal components: a primary component associated with collimator leaf 803, and a left component associated with collimator leaf 802. In the illustrated example, both components may have a value of +0, indicating that the center of Hall sensor head 910 is sensing the area marked “X/2.” In at least one example embodiment, a controller receiving the sensing signals generated by each Hall sensing head may use those sensing signals to adjust a previously determined position of each collimator leaf. It should be noted that in various example embodiments, sensing signals are not generated only at a stopping point of the collimator leaves. That is to say, as each collimator leaf moves along an axis of movement, the Hall sensing heads are continuously sensing and generating sensing signals. Thus, if a first-in-time sensing signal has a value of −1, and a second-in-time sensing signal has a value of +1, the controller will know that the sensor has crossed a Noth-South magnetic pole transition. By maintaining a record of pole transitions associated with each collimator leaf, the controller may determine a current position of each collimator leaf, and issue appropriate control signals to a motive device that moves the collimator leaves.

It will be appreciated that although the current example uses collimator leaves, other example embodiments may apply the same techniques to different types of movable parts.

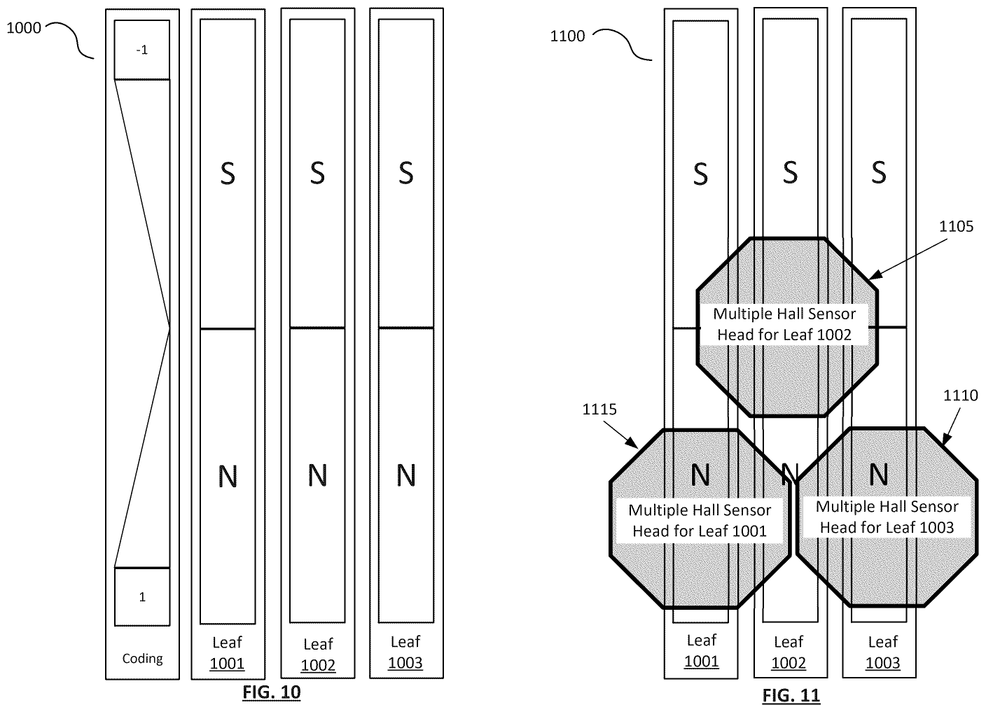

Referring next to FIG. 10 another collimator leaf arrangement 1000 will be discussed in accordance with various example embodiments, including single North and South pole pairs will be discussed in accordance with various example embodiments. In the illustrated example embodiments, collimator leaves 1001, 1002, and 1003 include a single magnet having North and South magnetic poles. In the collimator leaf arrangement 1000, the magnetic field may be represented by convention as lines of force flowing out of the North pole and into the South pole. The magnetic field lines may be assigned a positive value as they flow out of the North pole, and a negative value as they flow into the South pole. Note that the strength of the magnetic field is maximum at the poles, and is a minimum in the center of the magnet, where the North and South portions of the magnet meet. Thus, a magnetic sensor positioned close to the middle of the magnet will sense a weaker magnetic field towards the center of the magnet, and a stronger magnetic field at both the North and South poles. However, the direction of flow of the magnetic lines of force will be different at the South pole than at the North pole. Thus, a magnetic sensor may code the magnetic field sensed at the North pole as “+1”, the magnetic field sensed at the south pole as “−1,” and the magnetic field sensed at the transition point between North and South as “0.” Sensed magnetic fields between the North pole and the transition point may be assigned values between 0 and +1, based on the strength of the sensed magnetic field. Similarly, sensed magnetic fields between the South pole and the transition point may be assigned values between 0 and −1, based on the strength of the sensed magnetic field.

The term “coded,” as used in the description of FIGS. 10 and 11, refers to a value output by a magnetic field sensor when it senses a magnetic field associated with a portion of the collimator leaf. Furthermore, it will be appreciated that although the current example uses collimator leaves, other example embodiments may apply the same techniques to different types of movable parts.

Referring next to FIG. 11, another magnetic sensing arrangement 1100 will be discussed in accordance with various example embodiments. In the illustrated example embodiments, magnetic sensing arrangement 1100 includes multiple magnetic sensors, such as Hall sensor heads 1105, 1110, and 1115 positioned over collimator leaves 1001, 1002, and 1003. Hall sensor head 1105 may be used as the primary sensor for collimator leaf 1002, and to provide backup or redundant sensing for collimator leaves 1001 and 1003. Hall sensor head 1110 may be used as the primary sensor for collimator leaf 1003, and to provide backup or redundant sensing for collimator leaf 1002. Hall sensor head 1115 may be used as the primary sensor for collimator leaf 1001, and to provide backup or redundant sensing for collimator leaf 1002.

In at least one example embodiment, Hall sensor heads 1105, 1110, and 1115 detect magnetic field strengths of magnetic portions of collimator leaves 1001, 1002, and 1003 and code a sensed output component over a full range of values from “+1” and “−1.” The coded sensed values associated with the collimator leaves monitored by each Hall sensor head may be included as components in a composite signal transmitted to a controller. In some example embodiments, the components associated with each monitored collimator leaf may be sent as separate signals to the controller.

In an example of operation, a sensing signal generated by Hall sensor head 1105 may include three sensing signal components: a primary component associated with collimator leaf 1002, a left component associated with collimator leaf 1101, and a right component associated with collimator leaf 1103.

In the illustrated example, each of the three components may have a value of between about “0” indicating that the center of Hall sensor head 1105 is located over at or near a minimum magnetic field point at the transition from North to South.

A sensing signal generated by Hall sensor head 1110 may include two sensing signal components: a primary component associated with collimator leaf 1103, and a left component associated with collimator leaf 802. In the illustrated example, both components may have a value of approximately +0.5, indicating that the center of Hall sensor head 1110 is near the midpoint of the North magnetic portion of both collimator leaf 1002 and collimator leaf 1003.

A sensing signal generated by Hall sensor head 1115 may also include two sensing signal components: a primary component associated with collimator leaf 1002, and a right component associated with collimator leaf 1002. In the illustrated example, both components may have a value of approximately +0.5, indicating that the center of Hall sensor head 1115 is near the midpoint of the North magnetic portion of both collimator leaf 1001 and collimator leaf 1002.

Note that the same collimator leaf position is indicated by all three Hall sensing heads, even though the value of the sensing signal components varies between the different sensors. In at least one example embodiment, a controller receiving the sensing signals generated by each Hall sensing head may use those sensing signals to adjust a previously determined position of each collimator leaf, or to directly determine a current position of each collimator leaf. For example, a difference between a first magnetic field strength measurement and a second magnetic field strength measurement may be used to determine a change in position of a collimator leaf during a time interval between two readings. Alternatively, or in addition, a value of a field strength measurement may be used to directly indicate a position of the collimator leaf. It should be noted that in various example embodiments, sensing signals are not generated only at a stopping point of the collimator leaves. That is to say, as each collimator leaf moves along an axis of movement, the Hall sensing heads are continuously sensing and generating sensing signals. It will also be appreciated that although the current example refers to collimator leaves, other example embodiments may apply the same techniques to different types of movable parts.

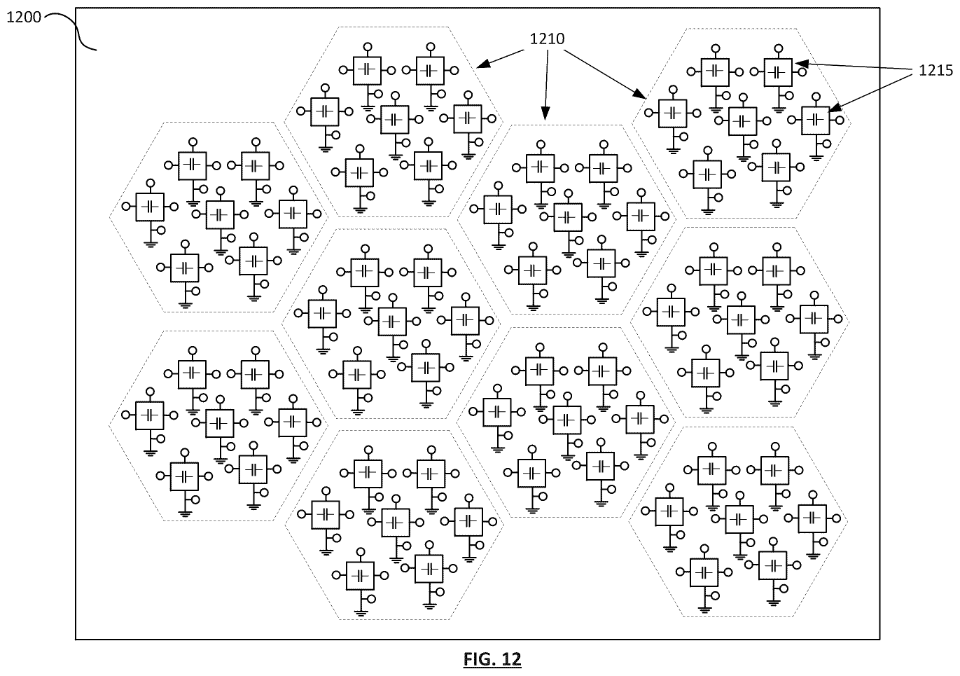

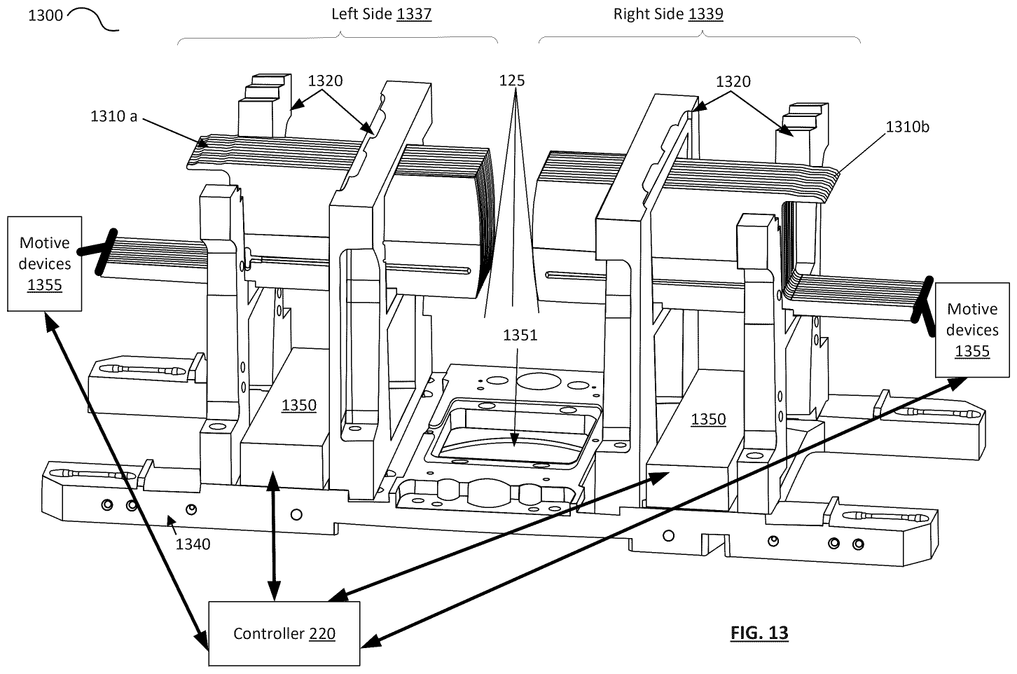

Referring next to FIG. 12, a capacitive sensor arrangement 1200 will be discussed in accordance with various example embodiments. The capacitive sensor arrangement 1200 includes multiple capacitive sensors, such as capacitive sensing heads 1210. Each of the capacitive sensing heads 1210 may include multiple capacitive sensing elements 1215. Various capacitive sensing techniques are known to those of ordinary skill in the art. In general, a capacitive sensor is an electronic device that is capable of detecting targets without direct physical contact. Capacitive sensors emit an electrical field from a sensing end of a capacitive sensor. An object, such as a moving part to be sensed moving through this electrical field, may be detected by the capacitive sensor or sensing element. In some example embodiments, applying different frequencies to different sensing elements in the capacitive sensor may allow differentiation between sensing signals associated with different collimator leaves or other movable parts. The redundant sensing functions discussed with respect to magnetic sensors above apply equally to the use of capacitive sensors.

Referring next to FIG. 13 a collimator box 1300 will be discussed in accordance with various example embodiments. In the illustrated example embodiments, multiple collimator leaves 1310a and 1310b within the collimator box 1300 are movably supported by a leaf holders 1320, which supports the collimator leaves 1310 and allows the collimator leaves to move independently from each other along parallel movement axes. The leaf holders 1320 are mounted on base 1340, which includes a target opening 1351 in the center. Position detection systems 1350 may also be mounted on base 1340 between leaf holders 1320. Position detection systems 1350 may include sensors, sensor elements, and/or associated sensing and signal transmission circuitry.

In an example of operation, a radiation beam 125 is directed between the collimator leaves 1310a on the left side 1337 of the collimator box 1300 and the collimator leaves 1310b on the right side 1339 of collimator box. The radiation beam 125 is directed between the collimator leaves and through target opening 1351. The collimator leaves 1310a and 1310b may be moved to block portions of the radiation beam 125 during treatment of a patient. The sensor assemblies transmit sensing signals indicating changes in position of collimator leaves 1310a and 1310b to controller 220, which in turn transmits control signals to motive devices 1355 that move collimator leaves in accordance with the control signals. The sensing and movement process may be iteratively repeated until each collimator leaf reaches a desired position.

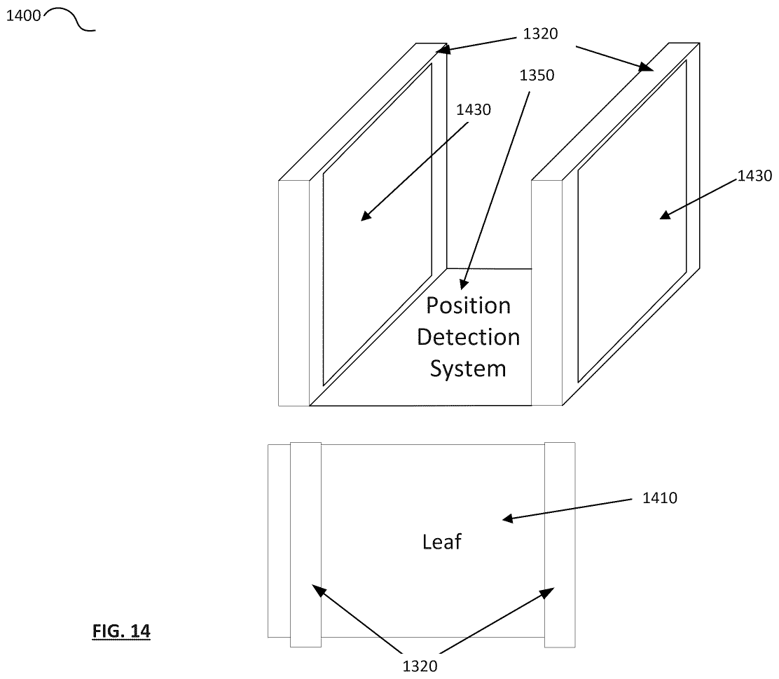

Referring next to FIG. 14, a generalized embodiment of leaf support system 1400 illustrating a location of a position detection system 1350 will be discussed in accordance with various example embodiments. In the illustrated example embodiment, the position detection system 1350 may be placed between leaf holders 1320. Note that although the position detection system 1350 is illustrated at the bottom of leaf support system 1400, the only requirement is that the position detection system 1350 be placed at a location that does not interfere with movement of each leaf 1410. In some example embodiments, the position detection system 1350 may be placed below, above, beside, or between leaves to facilitate sensing of positional changes of the individual leaves.

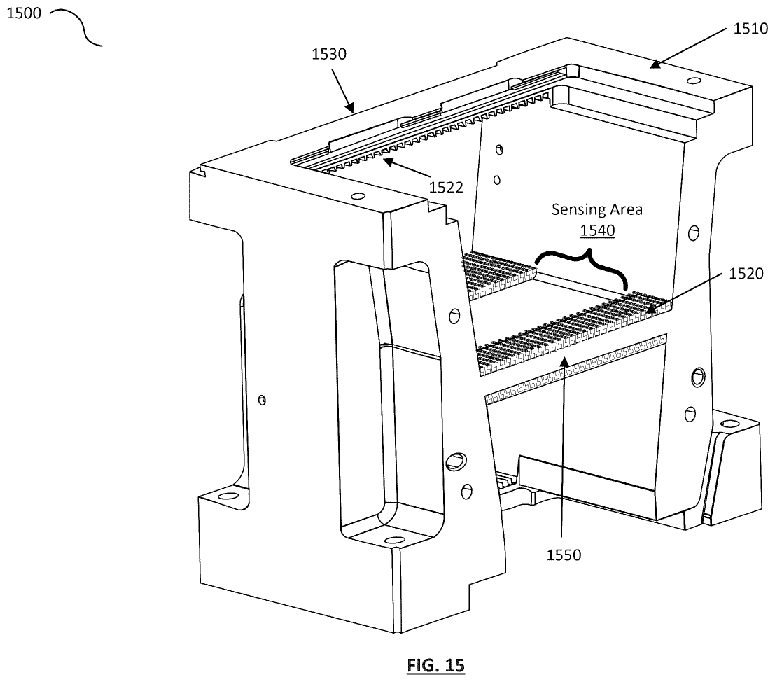

Referring next to FIG. 15 a leaf collimator support 1500 will be discussed in accordance with some example embodiments. Leaf collimator support 1500 includes a top portion 1510 including an upper crossbar 1530 including first guide teeth 1522, and a leaf support shelf 1550 having two sets of second guide teeth 1520 formed thereon. the upper crossbar 1530 the first guide teeth 1522, and the second guide teeth 1520, cooperate to keep collimator leaves aligned with an axis of movement. In the illustrated example embodiment, sensing area 1540 is provided for mounting sensors in close proximity to, but not contacting, collimator leaves supported by leaf collimator support 1500. Note that as illustrated, the portion of the collimator leaf used to block the radiation beam 125 (see FIGS. 1 and 13) protrudes to the left of the leaf collimator support 1500, while positional sensing may be performed in sensing area 1540. Other sensing areas may be used, but in at least one example embodiment sensing leaf positional changes using a portion of the leaf not primarily responsible for block portions of a radiation beam may help isolate sensors from radiation that in some circumstances might affect sensor operation.

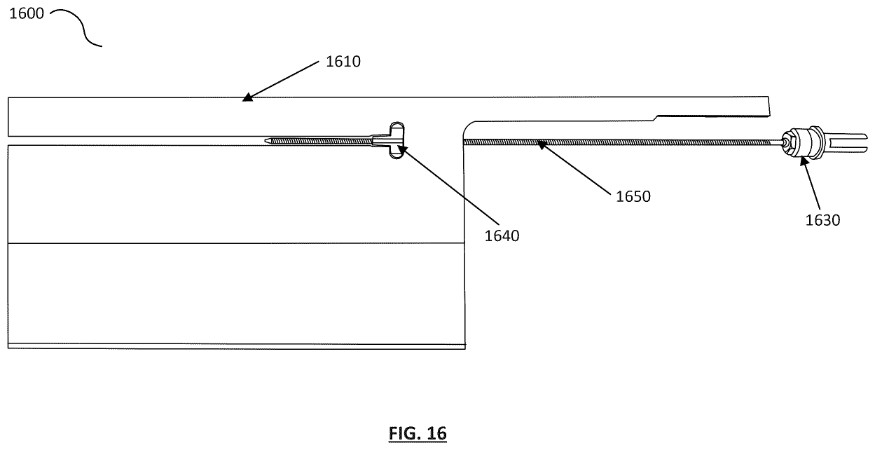

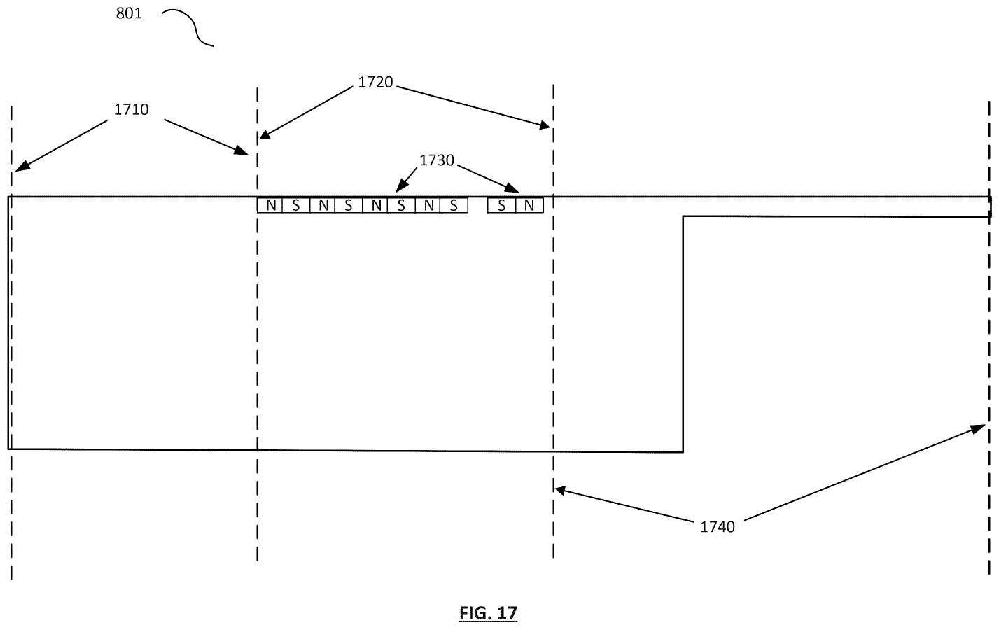

Referring next to FIG. 16, a collimator leaf motive system 1600 will be discussed in accordance with various example embodiments. In the illustrated example embodiments, motion may be imparted to collimator leaf 1610 by using a drive spindle 1650. As illustrated a drive nut 1640 is threaded onto drive spindle 1650. As drive spindle 1650 rotates, drive nut 1640 pushes (or pulls) collimator leaf 1610 along an axis of movement. Drive spindle 1650 includes a motor adaptor 1630, which is connected to an electric motor (not illustrated) controlled by control signals generated in response to sensing signals from any of various contactless sensors. In general, one drive spindle may be used for each of up to 60 collimator leaves. Each of the drive motors may be controlled individually.

It will be appreciated that although a spindle-drive mechanism is illustrated, other drive mechanisms known to those of ordinary skill in the art may be used. Such drive mechanisms may include, but are not limited to, friction wheels, gear-and-tooth arrangements, magnetic drives, or the like.

Referring next to FIG. 17 a collimator leaf 801 will be discussed in accordance with various example embodiments. In some example embodiments, the collimator leaf 801 includes multiple North and South pole pairs, which may be arranged as illustrated magnetized portion 1730. As illustrated in FIG. 17, the collimator leaf 801 may include three general areas. A blocking area 1710, which may be used to block portions of a radiation beam, sensing area 1720, and drive area 1740. Note that in the illustrated example embodiment, when collimator leaf 801 is in a fully retracted position (not illustrated), only a portion of magnetized portion 1730 may lie within sensing area 1720, but when collimator leaf 801 is in a fully extended position (as illustrated), magnetized portion 1730 does not extend into blocking area 1710. This arrangement may be used to prevent radiation from affecting the magnetized portion 1730 of collimator leaf 801. Note that in some example embodiments, magnetized portion 1730 may include individual magnets attached to or embedded within collimator leaf 801. In other example embodiments, at least a portion of collimator leaf 801 may be constructed of a ferromagnetic material to which a magnetic pattern has been applied. As will be subsequently illustrated, other arrangements of magnetic portion 1730 may be used. Furthermore, although magnetic portion 1730 is illustrated and discussed with reference to FIG. 17, optical, electrical, or other patterns may be arranged on collimator leaf 801 in conformance with the techniques used with magnetized portion 1730.

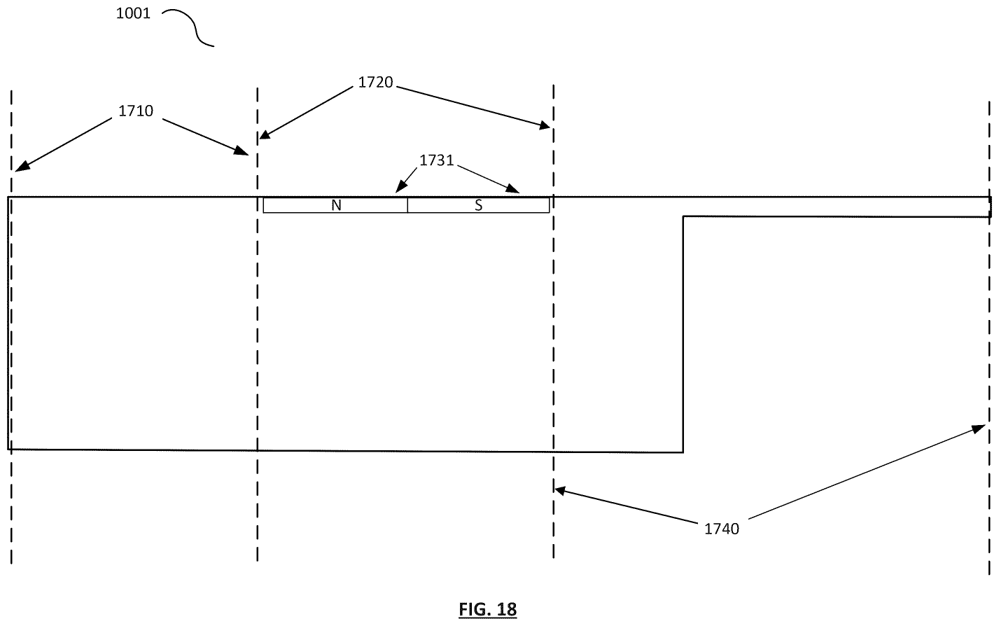

Referring next to FIG. 18, a collimator leaf 1001 will be discussed in accordance with various example embodiments. Collimator leaf 1001 is the same as collimator leaf 801, with the exception of magnetized portion 1731, which has only a single North-South pole pair as opposed to the multiple North-South pole pairs included in magnetized portion 1730 of collimator leaf 801.

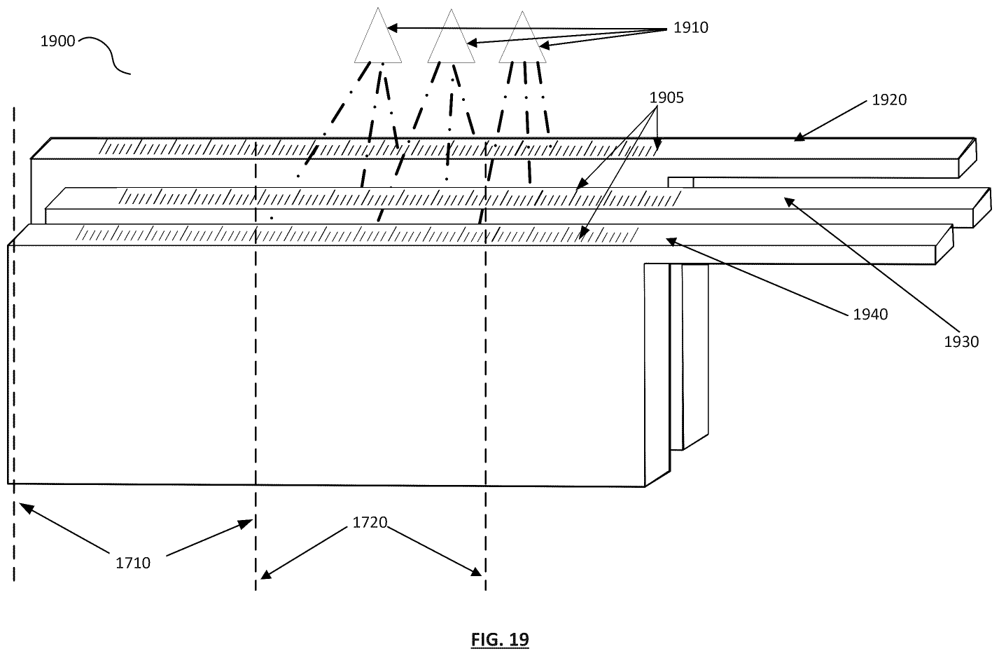

Referring next to FIG. 19, a sensing arrangement 1900 will be discussed in accordance with various example embodiments. In the illustrated example embodiments, sensing arrangement 1900 includes collimator leaves 1920, 1930, and 1940. Each of the collimator leaves 1920, 1930, and 1940 includes scales 1905 used for optical position sensing in conjunction with optical sensors 1910. As with the example embodiments discussed with reference to FIGS. 17 and 18, sensing may occur within sensing area 1720 of each of the collimator leaves. Unlike the example embodiments of FIGS. 17 and 18, the scales 1905 are unlikely to be affected by radiation, and may be extended even into the blocking area 1710. Additionally, in some example embodiments optical sensing may be performed outside of sensing area 1720.

Optical sensors 1910 may include any of various optical sensors known to those of ordinary skill in the art. In general, an optical sensor may include photosensitive semiconductor device that generates a signal in response to impingement of light. Detectors using one or mor different wavelengths of light may be used. In some example embodiments, a light source used in conjunction with an optical sensor may include a laser producing a wavelengths of light corresponding to the optical sensor. In some example embodiments, optical sensing may use laser triangulation or another optical sensing technique that does not rely on scales 1905 for operation. Each optical sensor may be used to monitor more than one collimator leaf or other moving part, thus providing redundancy of monitoring.

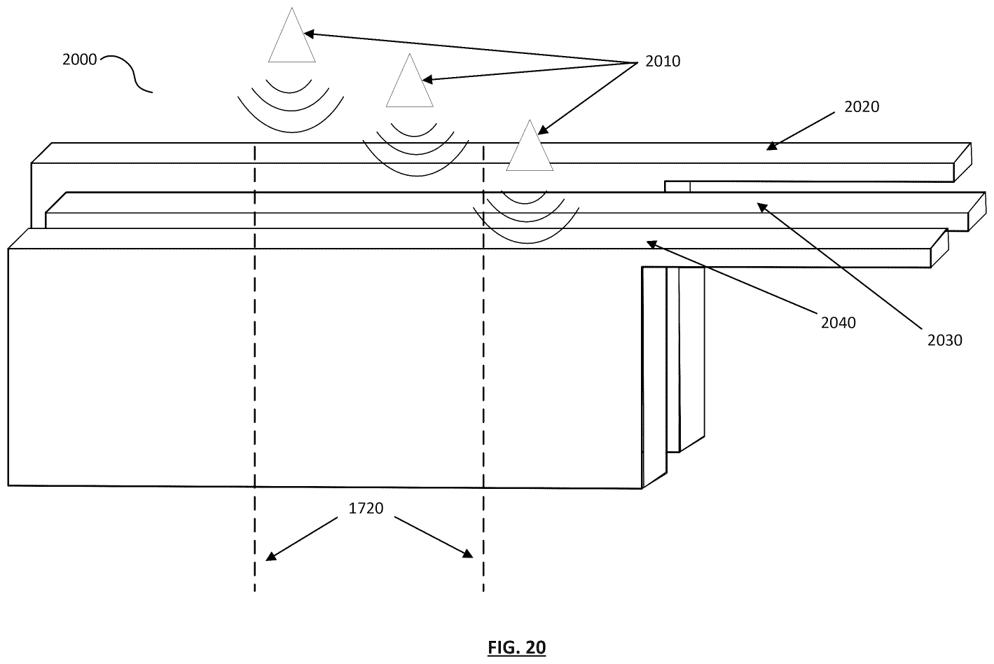

Referring next to FIG. 20 is a sensing system using 2000 reflected-energy sensors 2010, will be discussed in accordance with various example embodiments. The reflected-energy sensors 2010 may include acoustic sensors, optical sensors, or the like. Reflected-energy sensors may include their own energy generators, for example acoustic or light sources. In other example embodiments, one or more sources of energy external to the reflected-energy sensors 2010 may be used. Reflected-energy sensing may take place within sensing area 1720, or in other areas of collimator leaves 2020, 2030, and 2040. In the illustrated example embodiment, the reflected-energy sensors 2010 do not rely upon markings or other modifications to collimator leaves 2020, 2030, or 2040, although such markings or modifications may be used in conjunction with reflected-energy sensors 2010.

As with other types of sensors discussed herein, the reflected-energy sensors 2010 provide contactless, redundant sensing of moving parts, such as collimator leaves 2020, 2030 and 2040. In particular each of the reflected-energy sensors 2010 may be used to sense positional information related to multiple moving parts, and send sensing signal to a controller which controls movement of the multiple moving parts based on the sensing signals received from the sensors.

Various non-limiting illustrative embodiments are disclosed herein. Illustrative embodiment 1, includes a position control system comprising a plurality of contactless sensors configured to generate sensing signals indicating sensed movement of a plurality of parts configured to travel along a plurality of axes, and to transmit the sensing signals to a controller. The controller includes a processor and associated memory configured to cause the position control system to receive a first sensing signal from a first contactless sensor, the first sensing signal indicating sensed movement of particular parts included in a first subset of the plurality of parts; receive a second sensing signal from a second contactless sensor, the second sensing signal indicating sensed movement of a second subset of the plurality of parts, the second subset of the plurality of parts including at least one part included in both the first subset and the second subset; determine a first sensed position of the at least one part included in both the first subset and the second subset based on the first sensing signal; determine a second sensed position of the at least one part included in both the first subset and the second subset based on the second sensing signal; and generate a control signal configured to control movement of the at least one part included in both the first subset and the second subset based on the first sensed position and the second sensed position.

Illustrative embodiment 2 includes the position control system of illustrative embodiment 1, wherein the plurality of contactless sensors are configured to sense a movement of a plurality of medical device parts, and wherein the control system is configured to control the movement of the plurality of medical device parts based on the sensed movement of the plurality of medical device parts.

Illustrative embodiment 3 includes the position control system of illustrative embodiment 2, wherein the plurality of contactless sensors are configured to sense a movement of a plurality of collimator leaves included in a multi-leaf collimator, and the control system is configured to control a movement of each individual collimator leaf of the plurality of collimator leaves based on the sensed movement of the plurality of collimator leaves.

Illustrative embodiment 4 includes the position control system of any of illustrative embodiments 1-3, wherein the plurality of contactless sensors include a plurality of magnetic field sensors, the plurality of magnetic field sensors configured to magnetically sense movement of overlapping subsets of the plurality of parts.

Illustrative embodiment 5 includes the position control system illustrative embodiment 4, wherein each of the plurality of parts includes at least one magnetic portion configured with magnetic poles along an axis of movement.

Illustrative embodiment 6 includes the position control system of illustrative embodiment 4, wherein at least one of the plurality of magnetic field sensors is configured to detect a magnetic maxima and a magnetic minima.

Illustrative embodiment 7 includes the position control system of illustrative embodiment 4, wherein at least one of the plurality of magnetic field sensors is configured to detect changes in a magnetic field polarity.

Illustrative embodiment 8 includes the position control system of any of illustrative embodiments 1-7, wherein the plurality of contactless sensors include rotational sensors.

Illustrative embodiment 9 the position control system of any of illustrative embodiments 1-8, wherein the plurality of contactless sensors include linear sensors.

Illustrative embodiment 10 the position control system of any of illustrative embodiments 1-9, wherein the plurality of contactless sensors include optical sensors.

Illustrative embodiment 11 the position control system of illustrative embodiment 10, further comprising a plurality of lasers configured to illuminate the plurality of parts, wherein the optical sensors are configured to receive light reflected by the plurality of parts, and generate sensing signals based the light reflected by the plurality of parts, and the controller is configured to determine at least one of the first sensed position and the second sensed position based on the light received at the optical sensors.

Illustrative embodiment 12 includes the position control system of any of illustrative embodiments 1-11, wherein the plurality of contactless sensors include capacitive sensors.

Illustrative embodiment 13 includes the position control system of any of illustrative embodiments 1-12, further including a carriage box; a plurality of collimator leaves moveably mounted in the carriage box; a plurality of drives coupled to individual collimator leaves of the plurality of collimator leaves, the plurality of drives configured to independently move the individual collimator leaves along a plurality of parallel axes; and a plurality of contactless sensors mounted to the carriage box and configured to generate a first sensing signal indicating a sensed movement of a first subset of the plurality of collimator leaves, generate a second sensing signal indicating a sensed movement of a second subset of the plurality of collimator leaves, the second subset of the plurality of collimator leaves including at least one collimator leaf included in both the first subset and the second subset, and transmit the first sensing signal and the second sensing signal to a controller, the controller configured to control movement of the at least one collimator leaf included in both the first subset and the second subset based on the first sensing signal and the second sensing signal.

Illustrative embodiment 14 includes method comprising: sensing using a plurality of contactless sensors changes in positions of a plurality of parts configured to travel along a plurality of axes, the sensing including redundantly sensing positions of each of the plurality of parts by using at least two sensors to sense changes in positions of particular parts included in overlapping subsets of the plurality of parts; generating a first sensing signal indicating sensed movement of a first subset of the plurality of parts; generating a second sensing signal indicating sensed movement of a second subset of the plurality of parts, the second subset including at least one part also included in the first subset; and transmitting the first sensing signal and the second sensing signal to a controller configured to control movement of the at least one part included in both the first subset and the second subset based on the first sensing signal and the second sensing signal.

Illustrative embodiment 15 includes the method of illustrative embodiment 14 implemented using the position control system of any of illustrative embodiments 1-13.

As discussed herein, the terminology “one or more” and “at least one” may be used interchangeably. Furthermore, although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of this disclosure. As used herein, the term “and/or,” includes any and all combinations of one or more of the associated listed items.

When an element is referred to as being “connected,” or “coupled,” to another element, it may be directly connected or coupled to the other element, or more intervening elements may be present. By contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Specific details are provided in the preceding description to provide a thorough understanding of example embodiments. However, it will be understood by one of ordinary skill in the art that example embodiments may be practiced without these specific details. For example, systems may be shown in block diagrams so as not to obscure the example embodiments in unnecessary detail. In other instances, well-known processes, structures, and techniques may be shown without unnecessary detail in order to avoid obscuring example embodiments.

As discussed herein, illustrative embodiments have been described with reference to acts and symbolic representations of operations (e.g., in the form of flow charts, flow diagrams, data flow diagrams, structure diagrams, block diagrams, etc.) that may be implemented as program modules or functional processes include routines, programs, objects, components, data structures, etc., that perform particular tasks or implement particular abstract data types and may be implemented using existing hardware at, for example, existing user equipment or other network elements and/or hardware. Such existing hardware may be processing or control circuitry such as, but not limited to, one or more processors, one or more Central Processing Units (CPUs), one or more controllers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUs), one or more microprocessors, one or more Application Specific Integrated Circuits (ASICs), or any other device or devices capable of responding to and executing instructions in a defined manner.

Although a flow chart may describe the operations as a sequential process, many of the operations may be performed in parallel, concurrently, or simultaneously. In addition, the order of the operations may be re-arranged. A process may be terminated when its operations are completed, but may also have additional steps not included in the figure. A process may correspond to a method, function, procedure, subroutine, subprogram, etc. When a process corresponds to a function, its termination may correspond to a return of the function to the calling function or the main function.

As disclosed herein, the term “storage medium,” “computer readable storage medium” or “non-transitory computer readable storage medium” may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing and/or containing, instruction(s) and/or data.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a computer readable storage medium. When implemented in software, a processor or processors will perform the necessary tasks. For example, as mentioned above, according to one or more example embodiments, at least one memory may include or store computer program code, and the at least one memory and the computer program code may be configured to, with at least one processor, cause a network element or network device to perform the necessary tasks. Additionally, the processor, memory, and example algorithms, encoded as computer program code, serve as means for providing or causing performance of operations discussed herein.

A code segment of computer program code may represent a procedure, function, subprogram, program, routine, subroutine, module, software package, class, or any combination of instructions, data structures or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable technique including memory sharing, message passing, token passing, network transmission, etc.

The terms “including” and/or “having,” as used herein, are defined as comprising (i.e., open language). The term “coupled,” as used herein, is defined as connected, although not necessarily directly, and not necessarily mechanically. Terminology derived from the word “indicating” (e.g., “indicates” and “indication”) is intended to encompass all the various techniques available for communicating or referencing the object/information being indicated. Some, but not all, examples of techniques available for communicating or referencing the object/information being indicated include the conveyance of the object/information being indicated, the conveyance of an identifier of the object/information being indicated, the conveyance of information used to generate the object/information being indicated, the conveyance of some part or portion of the object/information being indicated, the conveyance of some derivation of the object/information being indicated, and the conveyance of some symbol representing the object/information being indicated.

According to example embodiments, user equipment, other network elements, or the like, may be (or include) hardware, firmware, hardware executing software or any combination thereof. Such hardware may include processing or control circuitry such as, but not limited to, one or more processors, one or more CPUs, one or more controllers, one or more ALUs, one or more DSPs, one or more microcomputers, one or more FPGAs, one or more SoCs, one or more PLUs, one or more microprocessors, one or more ASICs, or any other device or devices capable of responding to and executing instructions in a defined manner.

Benefits, other advantages, and solutions to problems have been described above with regard to specific embodiments of the invention. However, the benefits, advantages, solutions to problems, and any element(s) that may cause or result in such benefits, advantages, or solutions, or cause such benefits, advantages, or solutions to become more pronounced are not to be construed as a critical, required, or essential feature or element of any or all the claims.

What is claimed is:

1. A position control system comprising:
a plurality of contactless sensors configured to
generate sensing signals indicating sensed movement of a plurality of parts configured to travel along a plurality of axes, and
transmit the sensing signals to a controller; and
the controller including a processor and associated memory configured to cause the position control system to
receive a first sensing signal from a first contactless sensor, the first sensing signal indicating sensed movement of particular parts included in a first subset of the plurality of parts,
receive a second sensing signal from a second contactless sensor, the second sensing signal indicating sensed movement of a second subset of the plurality of parts, the second subset of the plurality of parts including at least one part included in both the first subset and the second subset,
determine a first sensed position of the at least one part included in both the first subset and the second subset based on the first sensing signal,
determine a second sensed position of the at least one part included in both the first subset and the second subset based on the second sensing signal, and
generate a control signal configured to control movement of the at least one part included in both the first subset and the second subset based on the first sensed position and the second sensed position.

2. The position control system of claim 1, wherein
the plurality of contactless sensors are configured to sense a movement of a plurality of medical device parts, and
the control system is configured to control the movement of the plurality of medical device parts based on the sensed movement of the plurality of medical device parts.

3. The position control system of claim 2, wherein
the plurality of contactless sensors are configured to sense a movement of a plurality of collimator leaves included in a multi-leaf collimator, and
the control system is configured to control a movement of each individual collimator leaf of the plurality of collimator leaves based on the sensed movement of the plurality of collimator leaves.

4. The position control system of claim 1, wherein:
the plurality of contactless sensors include a plurality of magnetic field sensors, the plurality of magnetic field sensors configured to magnetically sense movement of overlapping subsets of the plurality of parts.

5. The position control system of claim 4, wherein:
each of the plurality of parts includes at least one magnetic portion configured with magnetic poles along an axis of movement.

6. The position control system of claim 4, wherein:
at least one of the plurality of magnetic field sensors is configured to detect a magnetic maxima and a magnetic minima.

7. The position control system of claim 4, wherein:
at least one of the plurality of magnetic field sensors is configured to detect changes in a magnetic field polarity.

8. The position control system of claim 1, wherein
the plurality of contactless sensors include rotational sensors.

9. The position control system of claim 1, wherein
the plurality of contactless sensors include linear sensors.

10. The position control system of claim 1, wherein
the plurality of contactless sensors include optical sensors.

11. The position control system of claim 10, further comprising:
a plurality of lasers configured to illuminate the plurality of parts, wherein
the optical sensors are configured to receive light reflected by the plurality of parts, and generate sensing signals based the light reflected by the plurality of parts, and
the controller is configured to determine at least one of the first sensed position and the second sensed position based on the light received at the optical sensors.

12. The position control system of claim 1, wherein
the plurality of contactless sensors include capacitive sensors.

13. A leaf collimator comprising:
a carriage box;
a plurality of collimator leaves moveably mounted in the carriage box;
a plurality of drives coupled to individual collimator leaves of the plurality of collimator leaves, the plurality of drives configured to independently move the individual collimator leaves along a plurality of parallel axes; and
a plurality of contactless sensors mounted to the carriage box and configured to
generate a first sensing signal indicating a sensed movement of a first subset of the plurality of collimator leaves,
generate a second sensing signal indicating a sensed movement of a second subset of the plurality of collimator leaves, the second subset of the plurality of collimator leaves including at least one collimator leaf included in both the first subset and the second subset, and
transmit the first sensing signal and the second sensing signal to a controller, the controller configured to control movement of the at least one collimator leaf included in both the first subset and the second subset based on the first sensing signal and the second sensing signal.

14. The leaf collimator as in claim 13, wherein:
the plurality of contactless sensors include a plurality of magnetic field sensors, the plurality of magnetic field sensors configured to magnetically sense a movement of overlapping subsets of the plurality of collimator leaves.

15. The leaf collimator as in claim 14, wherein:
each of the plurality of collimator leaves includes at least one magnetic portion configured with magnetic poles along an axis of movement.

16. The leaf collimator as in claim 14, wherein the plurality of magnetic field sensors are configured to:
detect changes in a polarity of the magnetic fields; and
output polarity sensor signals indicating the changes in the polarity of the magnetic fields to a controller configured to determine positions of each individual collimator leaf based on the changes in the polarity of the magnetic fields associated with each individual collimator leaf.

17. The leaf collimator as in claim 14, wherein:
the at least one of the plurality of magnetic field sensors is configured to detect one or more of a magnetic maxima, a magnetic minima, or changes in a magnetic field polarity.

18. The leaf collimator as in claim 13, wherein:
the plurality of contactless sensors include one or more of rotational sensors, linear sensors, optical sensors, or capacitive sensors.

19. A method comprising:
sensing using a plurality of contactless sensors changes in positions of a plurality of parts configured to travel along a plurality of axes, the sensing including redundantly sensing positions of each of the plurality of parts by using at least two sensors to sense changes in positions of particular parts included in overlapping subsets of the plurality of parts;
generating a first sensing signal indicating sensed movement of a first subset of the plurality of parts;
generating a second sensing signal indicating sensed movement of a second subset of the plurality of parts, the second subset including at least one part also included in the first subset; and
transmitting the first sensing signal and the second sensing signal to a controller configured to control movement of the at least one part included in both the first subset and the second subset based on the first sensing signal and the second sensing signal.

20. The method as in claim 19, further comprising:
wherein the plurality of parts includes collimator leaves, and
detecting positions of at least two individual collimator leaves using a particular contactless sensor associated with the at least two individual collimator leaves as the at least two individual collimator leaves move along respective parallel axes.
